SEQUENCE LISTING (4 sequences)

SEQ ID NO: 1 - DNA, 20 bp, Artificial Sequence
  DNA Primer
ctaagacctgtggaatgggc

SEQ ID NO: 2 - DNA, 21 bp, Artificial Sequence
  DNA Primer
ctcaaagagttcattgccccc

SEQ ID NO: 3 - DNA, 20 bp, Artificial Sequence
  DNA Primer
accacagtccatgccatcac

SEQ ID NO: 4 - DNA, 20 bp, Artificial Sequence
  DNA Primer
tccaccaccctgttgctgta

(19) United States
(12) Patent Application Publication
(10) Pub. No.: US 2010/0130604 A1
(43) Pub. Date: May 27, 2010

(54) USE OF ROSMARINIC ACID IN MANUFACTURE OF MEDICAMENTS FOR TREATING OR PREVENTING HEPATIC AND RENAL DISEASES

(75) Inventors: Guisheng Li, Shandong (CN); Wanglin Jiang, Shandong (CN); Guiwu Qu, Shandong (CN); Jingwei Tian, Shandong (CN)

Correspondence Address: TUROCY & WATSON, LLP, 127 Public Square, 57th Floor, Key Tower, CLEVELAND, OH, 44114, US

(73) Assignee: SHANGDONG LUYE NATURAL DRUG RESEARCH AND DEVELOPMENT CO., LTD

(21) Appl. No.: 12/306,893

(22) PCT Filed: Jun. 29, 2007
(86) PCT No.: PCT/CN2007/002041

§ 371 Date: Sep. 2, 2009

(30) Foreign Application Priority Data
Jun. 29, 2006 (CN) 200610045376.X

(51) Int. Cl. A61K 31/216; C07C 69/73

(52) U.S. Cl. 514/533; 560/61

(57) ABSTRACT

The present invention demonstrates through many experiments that Rosmarinic acid (RA) can inhibit the expression of connective tissue growth factor (CTGF) and therefore is useful for the prevention or treatment of hepatofibrosis and nephrofibrosis. Based on this, the present invention provides novel uses of RA and pharmaceutical compositions comprising the same in the manufacture of medicaments useful for the prevention or treatment of chronic hepatitis, CRF, and diabetic nephropathy.

### TECHNICAL FIELD

The present invention relates to the medical use of known compounds, in particular, to the use of rosmarinic acid (RA) in the inhibition of the development and progress of hepatofibrosis or nephrofibrosis and therefore in the manufacture of medicaments for preventing or treating chronic hepatitis, chronic renal failure (CRF), and diabetic nephropathy; in particular, to the use of RA in the inhibition of the development and progress of hepatofibrosis or nephrofibrosis through inhibiting the expression of connective tissue growth factor (CTGF) and therefore in the manufacture of medicaments for preventing or treating chronic hepatitis, CRF, and diabetic nephropathy.

### BACKGROUND OF THE INVENTION

Hepatofibrosis is an important pathological feature in chronic hepatitis. Viruses, ethanol, and autoimmune diseases among others can all cause hepatocellular necrosis, regeneration, and continuous proliferation of fiber, which eventually lead to hepatocirrhosis. Now it is clear that hepatofibrosis is reversible while hepatocirrhosis is not. Prevention and treatment of the former are, therefore, of great significance in the treatment of chronic liver diseases.

Diabetic nephropathy is one of the most important complications of diabetes mellitus. Early pathological changes of the renal tissue include glomerular hypertrophy, extracellular matrix (ECM) accumulation, and thickening of glomerular basement membrane. Diffuse glomerular sclerosis occurs in advanced stages, leading to CRF. CRF refers to a clinical syndrome featuring a series of symptoms or a metabolic disorder caused by renal dysfunction as a result of primary or secondary chronic renal diseases. Nephrofibrosis (including renal interstitial fibrosis and glomerular sclerosis) is a common pathological feature of all renal diseases when they progress into the terminal phase. Pathogenesis of nephrofibrosis is rather complicated, involving many factors which mainly include proliferation and activation of ECM cell producing cells, vasoactive substances, cytokines, and imbalances between ECM production and degradation.

Connective tissue growth factor (CTGF) is one of the very important cellular growth factors as it regulates cell differentiation and proliferation, as well as extracellular matrix components. CTGF is considered to be closely related to transforming growth factor β1 (TGFβ1) in that CTGF is a downstream signal mediator in TGF-β's pro-fibrogenesis activity and mediates TGF-β's induction of cell proliferation and ECM production. Abnormal expression of CTGF contributes significantly to the development and progress of fibrosis in various organs.

Rosmarinic acid (RA) is also known as R(+)2-{[3-(3,4-dihydroxylphenyl-(oxo-2-propenyl)oxy]3,4-dihydroxylphenylpropionic acid]}. RA is a water soluble polyphenolic compound. It displays various activities such as anti-inflammatory, anti-oxidative, immunosuppressive, anti-thrombotic, and anti-platelet aggregation activity [Liu Y X and Ji Z Z, Progress in Pharmacological Research on Rosmarinic Acid, World Phytomedicines, 1993, 8(6): 248˜251; Chen S Z, Fu Y P et al., Effect of Rosmarinic Acid on Formation of Neutrophil Free Radicals and Release of Lysosome in Rats, Acta Pharmaceutica Sinica, 1999, 34(12): 881˜885; Peake P. W. Pussell B. A, Martyn P, et al. The inhibitory effect of Rosmarinic acid on complement involves the C5 convertase. Int J Immuno pharmacol 13:853˜857, 1997; Zou Z W, Xu L N, and Tian J Y, Antithrombotic and Anti-platelet Aggregation Effect of Rosmarinic Acid, Acta Pharmaceutica Sinica, 1993, 28(4): 241˜245]. However, RA's pharmacological effects in the prevention or treatment of hepatofibrosis and nephrofibrosis have not yet been reported.

### SUMMARY OF THE INVENTION

The present invention provides the use of RA in the manufacture of medicaments for inhibiting CTGF expression.

The present invention provides the use of RA in the manufacture of medicaments for preventing or treating hepatofibrosis.

The present invention provides the use of RA in the manufacture of medicaments for preventing or treating nephrofibrosis.

The present invention provides the use of RA in the manufacture of medicaments for preventing or treating chronic hepatitis.

The present invention provides the use of RA in the manufacture of medicaments for preventing or treating diabetic nephropathy.

The present invention provides the use of RA in the manufacture of medicaments for preventing or treating CRF.

When used in treating chronic hepatitis, CRF, and diabetic nephropathy, the dosages of the medicaments according to the present invention are in the range of 25-1,500 mg, preferably 25-750 mg when injected; 50 mg-3,000 mg, preferably 50-1,500 mg when taken orally.

The present invention further provides medicaments consisting of RA and pharmaceutically acceptable carriers or excipients. Said medicaments can be prepared as tablets, capsules, pills, solutions for injection, freeze-dried powers, or emulsions for injection, preferably as tablets, pills, or freeze-dried powders by conventional pharmaceutical methods.

The RA mentioned in the present invention can be prepared according to methods disclosed in Chinese patent application CN2005101311297, or purchased commercially.

The present inventors conducted a large number of studies and found that RA can prevent or treat hepatic or renal fibrosis through inhibiting CTGF expression. Based on these findings, the present inventors provide uses of RA in the inhibition of the development and progress of hepatofibrosis or nephrofibrosis through inhibiting the expression of CTGF and therefore in the manufacture of medicaments for preventing or treating chronic hepatitis, CRF, and diabetic nephropathy.

### THE DETAILED DESCRIPTION OF THE INVENTION

RA used in the following embodiments was prepared by Natural Drug Engineering, Technology, and Research Centre of Shandong Province according to methods provided in Example 1 of Chinese patent application CN2005101311297.

### Example 1

### Preparation of Freeze-Dried Powder of RA

To 50.0 g of RA, 2,000 ml of water for injection was added to dissolve it. Add NaOH until pH=5.5˜7.5. Add 8 g of mannitol, and stir until it completely dissolved. Pyrogen-free clear solution was obtained after ultrafiltration. 2 ml of the resulting solution was filled into each 10 ml tube vials and lyophilisation process was carried out to obtain lyophilised preparations with 50.0 mg of RA in each vial.

### Example 2

### Preparation of RA Tablets

100.0 g of RA, 35.0 g of sucrose, 40.0 g of lactose, and 23.0 g of Sodium Carboxymethyl Starch were mixed well and passed through 100-mesh sieve. 3% of PVP_{K30} aqueous solution was added as needed to make a damp mass, which was then granulated through 20-mesh sieve. After dried at 60° C. for 3 hours, the granules are passed through 18-mesh sieve. 2.0 g of magnesium stearate was added and mixed completely before compressed with shallow concavity. Tablets weighing 200 mg each were obtained.

RA used in the following examples was prepared by Natural Drug Engineering, Technology, and Research Centre of Shandong Province according to methods provided in Example 1 of Chinese patent application CN2005101311297.

### Test Example 1

### Effect of RA on CTGF Expression in Hepatic Stellar Cells (HSCs)

1 Materials

Dulbecco's modified Eagle's medium (DMEM) was purchased from Sigma. Neonatal bovine serum (NBS) was obtained from Hangzhou Sijiqing Biological Engineering Materials Co. Ltd. UNIQ-10 column RNA extraction kit, AMV first strand cDNA synthesis kit, instant PCR amplication kit, primers, diethyl phosphoryl cyanide (DEPC), ethidium bromide (EB), and DNA markers were all purchased from Shanghai Sangon Biological Engineering Technology & Services Co. Ltd. Monoclonal anti-CTGF antibody was from Santa Cruz Co. Ltd. Horseradish peroxidase (HRP) labeled rabbit-anti-goat secondary antibodies were purchased from Wuhan Boster Co. Ltd. All other reagents were imported or analytical pure according to Chinese standards.

Ordinary P×2 PCR apparatus was purchased from Thermo Hybaid, USA. Alphalmager 3400 Image Analysis Instrument was obtained from Alpha Innotech, USA. ELX800 ELISA Reader and ELX Automatic Plate Washer were obtained from Bio-Tek.

2 Methods and Results

Cells and experimental groups: HSC cell line was HSC-T6 (Shanghai Biosis Biotechnology Co. Ltd.), with the phenotype of active HSC. The cells were divided into groups for 0, 2.5, 5, 10, 20, and 40 μmol·L⁻¹ of RA. MTT method (Ye T and Liu X C, Effect of Pentoxifilline on Proliferation of Mesangial Cell Cultured in glucose-rich Medium and on CTGF Expression, Chinese Pharmacological Bulletin, 2004, 20(8): 883-885) was used to determine the effective RA concentration. Effects of RA on HSC-T6 proliferation and CTGF expression were examined.

2.1 Preparation of RA Stock Solution

A 1000 mg/L stock solution of RA in DMEM medium was prepared, and filtered through 0.22 μm microfilter to remove bacteria. The stock was then aliquoted in appropriate containers and stored at −20° C. in dark. It is good for use within 2 weeks. DMEM supplemented with 2% fetal bovine serum (FBS) was used to dilute the stock to desired concentrations before use.

2.2 HSC-T6 Cell Culture and Passage

HSC-T6 cells were cultured in DMEM supplemented with 100 U·mL⁻¹ penicillin, 100 U·mL⁻¹ streptomycin, and 10% FBS at 37° C., 5% CO₂, and saturated humidity. Medium was changed every other day. The culture was digested with 0.002% EDTA and 0.25% trypsin at 80-90% confluence. And the cells were passaged at 1:4.

2.3 Effect of RA on HSC-T6 Cell Growth

HSC-T6 cells at logarithmic phase after thawed were digested with trypsin and seeded to 96-well plate in DMEM supplemented with 5% FBS at the density of 1×10⁴. After 24 hours culture, different concentrations of RA were added according to table 1. Each concentration of RA was added to 6 wells. Blank wells were designed to serve as control. After 48 hours of incubation, the culture media were discarded, and 20 μL of 0.5% MTT was added to each well. The supernatant was discarded after 4 hours of incubation, and 200 mL of dimethyl sufoxide (DMSO) was added to each well to obtain cell lysate, which was then shaken well before detected for OD value on ELISA reader (492 nm). Cell growth inhibition rates were calculated.

As shown in table 1, different concentrations of RA significantly inhibited HSC-T6 proliferation compared to the control group (no RA). Also, the inhibitory effect of RA became more obvious with time.

TABLE 1
Inhibitory effect of RA on HSC-T6 cell growth ( x̅ ± SD)

| | Inhibition rate (%) | | |
|---|---|---|---|
| Conc. (mg/L) | 12 h | 24 h | 48 h |
| 0 | 2.1 ± 1.0 | 2.4 ± 1.1 | 2.7 ± 1.0 |
| 0.1 | 9.8 ± 2.3** | 12.1 ± 2.2** | 14.4 ± 2.3** |
| 1 | 20.7 ± 2.0** | 23.0 ± 2.0** | 25.3 ± 2.1** |
| 10 | 37.2 ± 2.6** | 38.6 ± 1.9** | 38.9 ± 3.2** |
| 50 | 52.3 ± 4.7** | 54.6 ± 4.6** | 55.2 ± 3.5** |
| 100 | 57.2 ± 2.3** | 59.6 ± 2.1** | 61.8 ± 2.2** |

*P < 0.05 and
**P < 0.01 compared with the control group.

### 2.4 Total RNA Extraction

Total RNA was extracted from the cell lysate obtained in section 2.3 according the manufacturer's instructions with UNIQ-10 column total RNA extraction kit. A UV spectrophotometer was used to measure the concentration and purity. This was repeated three times and the total RNA concentration was calculated.

2.5 RT-PCR Detection of CTGF Expression in HSC

Reaction system: 1 μL of RT product; 10 μL of 2×PCR Master; 1 μL of each of sense primer and anti-sense primer; added double distilled water to 20 μL.

Sense primer: 5′-CTAAGACCTGTGGAATGGGC-3′;

Anti-sense primer: 5′-CTCAAAGAGTTCATTGCCCCC-3′; primer length of 383 bp;

Internal standard GAPDH (Kangcheng Bio-tech Inc.), with sense primer as 5′-ACCACAGTCCATGCCATCAC-3′, anti-sense primer as 5′-TCCACCACCCTGTTGCTGTA-3′, and length of 452 bp;

Reaction conditions: pre-denature at 94° C. for 2 min, denature at 94° C. for 45 s, anneal at 54.9° C. for 30 s, extend at 72° C. for 60 s, extend at 72° C. for 10 min after 35 cycles and detect with 1.2% agarose gel electrophoresis. Scan with gel image system and calculate the ratio of gray scale between CTGF and GAPDH as an indicator of CTGF expression.

According to the results from the MTT method, groups of 0, 10, and 50 mg/L RA were selected and subjected to RT-PCR. It showed that RA of 10 and 50 mg/L exerted significant inhibition on CTGF mRNA expression in HSC-T6.

TABLE 2
Effect of RA on CTGF mRNA expression in HSC-T6 ( x̅ ± SD)

| Conc. (mg/L) | CTGF mRNA expression rate (%) |
|---|---|
| 0 | 98.5 ± 2.4 |
| 10 | 71.2 ± 8.7** |
| 50 | 63.2 ± 9.2** |

*P < 0.05 and
**P < 0.01 compared with the control group.

### Test Example 2

### Effect of RA on CTGF Expression of Proximal Tubular Epithelial Cells (PTECs) Cultured in Glucose-Rich Medium

1 Materials

Dulbecco's modified Eagle's medium (DMEM) and Ponceau S were purchased from Sigma. Fetal bovine serum (FBS) was obtained from Hangzhou Sijiqing Biological Engineering Materials Co. Ltd. Monoclonal anti-CTGF antibody was from Santa Cruz Co. Ltd. All other reagents were imported or analytical pure according to Chinese standards.

ELX800 ELISA Reader and ELX Automatic Plate Washer were obtained from Bio-Tek, USA.

2 Methods and Results

2.1 Effect of RA on Proliferation of PTECs Cultured in Glucose-Rich Medium

A 1000 mg/L stock solution of RA in DMEM medium was prepared, and filtered through 0.22 μm microfilter to remove bacteria. The stock was then aliquoted in appropriate containers and stored at −20° C. in darkness. It is good for use within 2 weeks. DMEM supplemented with 2% fetal bovine serum (FBS) was used to dilute the stock to desired concentrations before use.

Remove frozen vials storing PTECs from liquid nitrogen, thaw in water bath at 37° C. for 5 min, centrifuge at 1000 r·min⁻¹ for 5 min, and discard the supernatant. Blow up the cells with culture medium and transfer them into flasks for static culture. The medium was DMEM supplemented with 10 v % FBS, 1×10⁵ U·L⁻¹ penicillin, 100 mg·L⁻¹ streptomycin. The cells are cultured at 37° C., 5% CO₂, and saturated humidity for 2-3 days before 0.25% trypsin were used to digest the cells which were then passaged.

PTEC cells at logarithmic phase were seeded to 96-well plate at the density of 1×10⁵/mL. The medium was replaced with serum-free DMEM 24 hours later and the cell culture was cultured statically for 24 hours. Discard the supernatant, dose the wells according to table 3: normal control group (NG, 5 mmol·L⁻¹), high glucose group (HG, 30 mmol·L⁻¹), NG+10 mg/L RA group, NG+50 mg/L of RA group, HG+10 mg/L of RA group, HG50 mg/L of RA group, with 6 wells in each group. Incubate for 72 hours respectively. Add 20 μL of MTT (5 mg·mL⁻¹) to each well 4 hours before the experiment is terminated. Remove supernatant 4 hours later, and 150 μL of DMSO was added to each well, shake for 10 min and use ELISA reader to determine OD value at 570 nm.

PTECs proliferate more after treatment with high level of glucose for 72 hours, compared to the normal control group (P<0.05). Certain concentrations of RA can inhibit PTECs proliferation induced by high level of glucose (P<0.05), and the results are shown in table 3.

TABLE 3
Effect of RA on PTECs proliferation ( x̅ ± SD)

| Group | OD value |
|---|---|
| Normal control group NG | 1.105 ± 0.187 |
| High glucose group HG | 1.817 ± 0.086* |
| NG + 10 mg/L of RA | 1.168 ± 0.106 |
| NG + 50 mg/L of RA | 1.052 ± 0.036 |
| HG + 10 mg/L of RA | 1.289 ± 0.041^{#} |
| HG + 50 mg/L of RA | 1.317 ± 0.077^{#} |

*P < 0.05 compared with the NG group;
^{#}P < 0.05 compared with the HG group.

2.2 Effect of RA on Intracellular CTGF Expression

Western blot method was used to detect intracellular CTGF expression. PTEC cells at logarithmic phase were seeded to 96-well plate at the density of 1×10⁵/mL. The medium was replaced with serum-free DMEM 24 hours later and the cell culture was cultured statically for 24 hours. Treat the wells as mentioned above. 72 hours later, cellular protein was extracted from the cell lysate according to modified Lowry method (Peterson G L. A simplification of the protein assay method of Lowry et al, which is more generally applicable. Anal Biochem, 1977, 83:346). 75 μg of total protein was loaded on each lane and all lanes were subject to 12% SDS-PAGE before the protein was transferred onto nitrocellulose membrane. Dye with Ponceau S (Sigma) to confirm transference and mark relative molecular weight standards. The membrane was then blocked in 5% solution of skimmed milk powder in TBST for 1 hour at room temperature. After washing, multiclonal goat-anti-rat anti-CTGF antibody (1:500, from Santa Cruz Co. Ltd.) was added and incubated overnight at 4° C. Then HRP-labeled anti-goat IgG secondary antibody (1:2 500, from Santa Cruz Co. Ltd.) was added and incubated at 37° C. for 1 hour. After rinsing, develop with 3,3-diaminobenzidine (DAB). Relative OD values of each band were analysed and the data are presented as χ̅±s. SPSS software was used in statistical analysis. ANOVA was applied for multi-group comparison.

Relative molecular weight of CTGF is 3.7×10⁴, all groups showed bands at the corresponding position, and the results are shown in table 4. Normally PTECs have weak expression of CTGF. In the HG group, intracellular CTGF expression was markedly upregulated. Compared with the HG group, intracellular CTGF expression in the 10 mg/L and 50 mg/L RA groups was significantly down-regulated, indicating RA's inhibitory effect on CTGF expression.

TABLE 4
Effect of RA on intracellular CTGF protein expression ( x̅ ± SD)

| Group | CTGF expression |
|---|---|
| Normal control group NG | 5145 ± 95 |
| High glucose group HG | 5944 ± 128* |
| HG + 10 mg/L of RA | 5244 ± 106^{#} |
| HG + 50 mg/L of RA | 5345 ± 95^{#} |

*P < 0.05 compared with the NG group;
^{#}P < 0.5 compared with the HG group.

### Test Example 3

### Treatment of Hepatofibrosis in Chronic Hepatitis with RA

3.1 Drugs and Reagents

RA freeze-dried powder was prepared according to the above example 1. RA tablets were prepared according to the above example 2.

Avandi™ (Rosiglitazone Maleate Tablet, Glaxo SmithKline).

RIA kits for hyaluronic acid (HA), laminin (LN), and type III collagen (PcIII) were purchased from Shanghai Haiyan Medical Centre; Hydroxyproline (HYP) detection kit was purchased from Nanjing Jiancheng Bio-engineering Institute.

Experimental animals: Male SPF Sprague Dawley rats weighing 150 g-200 g were provided by Experimental Animal Centre of Shangdong Luye Pharmaceuticals Co. Ltd. Animal certificate number is SYXK (Lu) 20030020.

3.2 Methods:

120 rats were randomly divided into 12 groups, with 10 in each group, namely, a normal control group, a model group, RA i.v. groups at 2.5 mg/kg, 5 mg/kg, 25 mg/kg, 75 mg/kg, and 150 mg/kg of RA, and RA p.o. groups at 2.5 mg/kg, 5 mg/kg, 25 mg/kg, 150 mg/kg, and 300 mg/kg of RA. Apart from the normal control group, all rats were s.c. injected 0.3 ml of 40% solution of CC1₄ in oil per 100 g of body weight once every 3 days for 6 weeks. Animals in the normal control group were s.c. injected 0.3 ml/100 g body weight of oil vehicle following the same schedule as the experimental groups. First dose was doubled for all animals. Six weeks later, drugs were continuously administered to animals in each group for 6 weeks. After the administration was completed, all animals were anaesthetised with i.p. injection of 20% ethyl carbamate solution (Beijing Tongxian District Yucai Fine Chemicals). Blood was collected from abdominal aorta. Hepatic lobule tissue was removed, part of which was fixed with 10% neutral foinialin solution and made into wax blocks within 24-48 hours. Histopathological examination was conducted with RE staining and fiber proliferation was scored as 0-4 levels (Li K, Zhao Y Z, and Zhu Q S et al, Effect of Ligustrazine on cardiac and hepatic SOD activity in old mice, Heilongjiang Medicine and Pharmacy, 1998, 21: 4-5). Serum HA, LN, Pc III levels and hepatic HYP level were determined according to the respective kit instructions.

3.3 Results

Pathology Examination:

Hepatic histology was normal in the control group. Obvious fibrosis was observed in the livers of animals in the model group at 12 weeks. All animals in RA treated groups had less severe fibrosis in their livers compared to the model group.

Light Microscopic Examination:

Liver tissue sections with regular HE staining and VG collagen staining showed liver steatosis and necrosis as well as inflammatory cell infiltration in the liver fibrosis model control group. Collagenous fibers deposited in the portal area and hyperplasia were observed in Hennys duct as well as fiber connective tissue. Fibrous septa was observed to be thicker and typical pseudolobule formed. Animals in the RA treated groups had less fiber connective tissue hyperplasia, thinner fibrous septa and less pseudolobule formation. Fibrosis in different groups was analysed with Rank-sum test and the results are summarised in table 5. I.v. dose of 2.5mg/kg, 5 mg/kg, 25 mg/kg, 75 mg/kg, and 150 mg/kg of RA and p.o. Dose of 5 mg/kg, 25 mg/kg, 150 mg/kg, and 300 mg/kg of RA can significantly reduce fibrosis.

Electronic Microscopic Examination:

In the normal control group, hepatocytes were closely linked each other, with typical distribution of all cellular organelles. Blood sinusoids are regularly aligned, and lipocytes with fat drops in the cytoplasm can be seen in Space of Disse. In the model group, typical hepatocyte damages can be observed in the liver tissue. Gaps between adjacent hepatocytes were widened, and hepatocytes became degenerative and necrotic with pycnosis. Fat drops of irregular size and distribution pattern appeared in the cytoplasm. Fibrosis of different degrees was observed in the liver tissue. Liver sinusoid capillarisation was observed and many fibroblasts (activated lipocytes) were seen in Space of Disse with a large amount of collagenous fibers depositing in surrounding areas as well as in the portal area. In the RA treated groups, hepatocyte damages were alleviated with gaps between cells thinner and fat droplets less in the cytoplasm. Intracellular structure approaches normal state. No obvious fibrosis and less collagenous fiber deposition and fibroblasts in Space of Disse and blood sinusoids were observed.

TABLE 5
Effect of RA on pathomorphism of hepatofibrosis in rats ( x̅ ± SD,
n = 10)

| | Dosage | | | | | | |
|---|---|---|---|---|---|---|---|
| Group | mg/kg | 0 | I | II | III | IV | T |
| Normal | — | 10 | 0 | 0 | 0 | 0 | — |
| control | | | | | | | |
| Model | NS | 0 | 0 | 1 | 4 | 5 | — |
| RA i.v. | 2.5 | 0 | 1 | 2 | 3 | 4 | 82* |
| | 5 | 0 | 1 | 2 | 6 | 1 | 81.5* |
| | 25 | 0 | 2 | 3 | 4 | 1 | 76* |
| | 75 | 0 | 3 | 2 | 5 | 0 | 71** |
| | 150 | 0 | 3 | 4 | 3 | 0 | 66** |
| RA p.o. | 2.5 | 0 | 1 | 2 | 3 | 4 | 95 |
| | 5 | 0 | 1 | 2 | 7 | 0 | 77* |
| | 25 | 0 | 3 | 2 | 5 | 0 | 71** |
| | 150 | 0 | 3 | 4 | 3 | 0 | 66** |
| | 300 | 0 | 2 | 5 | 2 | 1 | 66** |

*P < 0.05 and
**P < 0.01 compared with the model group.

TABLE 6
Effect of RA on HYP, HA, LN, and PcIII levels in rats with chronic hepatitis
( x̅ ± SD, n = 10)

| | Dosage | HYP | PCIII | HA | LN |
|---|---|---|---|---|---|
| Group | (mg/kg) | (μg/L) | (μg/L) | (μg/L) | (μg/L) |
| Normal | — | 760 ± 90** | 14.2 ± 2.8** | 40.7 ± 7.5** | 31.3 ± 6.9** |
| control | | | | | |
| Model | NS | 1869 ± 130 | 48.6 ± 8.5 | 140.4 ± 24.1 | 91.6 ± 19.4 |
| Rosiglitazone | 8 | 1259 ± 153** | 26.5 ± 9.6** | 88.9 ± 20.5** | 63.6 ± 16.8** |
| RA i.v. | 2.5 | 1657 ± 124* | 36.2 ± 7.8* | 111.2 ± 24.1* | 72.6 ± 19.0* |
| | 5 | 1630 ± 110* | 34.2 ± 7.6* | 106.3 ± 23.4* | 68.3 ± 17.9* |
| | 25 | 1578 ± 113** | 31.4 ± 7.8** | 97.5 ± 23.6** | 62.0 ± 20.0** |
| | 75 | 1278 ± 121** | 26.5 ± 9.1** | 77.9 ± 20.7** | 57.6 ± 16.8** |
| | 150 | 1217 ± 143** | 25.7 ± 7.8** | 74.6 ± 18.9** | 55.3 ± 14.6** |
| RA p.o. | 2.5 | 1724 ± 121 | 38.0 ± 7.5 | 113.8 ± 24.8 | 79.1 ± 19.4 |
| | 5 | 1614 ± 121* | 33.7 ± 7.3* | 104.3 ± 26.4* | 69.3 ± 16.9* |
| | 25 | 1535 ± 125** | 31.6 ± 7.2** | 97.0 ± 24.6** | 61.0 ± 20.6** |
| | 150 | 1299 ± 153** | 27.5 ± 9.6** | 84.9 ± 20.5** | 59.6 ± 16.8** |
| | 300 | 1250 ± 174** | 26.9 ± 10.5** | 80.7 ± 18.6** | 57.3 ± 18.6** |

*P < 0.05 and
**P < 0.01 compared with the model group.

As shown in table 6, i.v. dose of RA at 2.5mg/kg, 5 mg/kg, 25 mg/kg, 75 mg/kg, and 150 mg/kg and p.o. dose of RA at 5 mg/kg, 25 mg/kg, 150 mg/kg, and 300 mg/kg can all significantly reduce HA, LN, Pc III, and HYP levels (compared with the model group, p<0.05or 0.01).

### Test Example 4

### Effect of RA on Renal Interstitial Fibrosis in Rats with Unilateral Urethral Obstruction

4.1 Materials

RA formulations were prepared according the Examples 1 and 2.

Benazapril was purchased from Beijing Novartis Pharmaceuticals; HYP kit was purchased from Nanjing Jiancheng Bio-engineering Institute; fibronectin (FN) kit was provided by Shanghai Institute of Biological Products.

Experimental animals: Male SPF Sprague Dawley rats weighing 220 g-250 g were provided by Experimental Animal Centre of Shangdong Luye Pharmaceuticals Co. Ltd. Animal certificate number is SYXK (Lu) 20030020.

4.2 Methods and Results:

130 rats were randomly divided into 13 groups, namely, a sham operation group, a model group, benazapril p.o. Group at 10 mg/kg, RA i.v. groups at 2.5 mg/kg, 5 mg/kg, 25 mg/kg, 75 mg/kg, and 150 mg/kg of RA, and RA p.o. groups at 2.5 mg/kg, 5 mg/kg, 25 mg/kg, 150 mg/kg, and 300 mg/kg of RA. The animals were acclimated for a week before they were all anaesthetised with an i.p. dose of 10% chloral hydrate at 3.0mL/kg and placed on the operating table in right lateral position. The rats were shaved and the operating area was disinfected using iodine tincture and 75% ethanol. The left urethra was exposed and isolated after a left lateral incision was made on the abdominal area and skin, muscles and all other layers of the abdominal wall were incised sequentially. In the sham group, the left urethra was only isolated without being ligated or cut off. In all other animals, the left urethra was ligated with No. 4 suture and obstructed followed by closure of the abdominal wall layer by layer. The animals were anaesthetised again with 10% chloral hydrate 10 days later, blood was collected and serum was separated to determine FN levels. The left kidney was removed after thorough rinsing and then fixed in 4% paraformaldehyde buffer. HYP level was determined according to instructions provided with the kit.

Regular Pathology Examination:

□ Visual Check:

In the sham operation group, the kidney was blood-red and smooth on the surface. Kidney peplos were glossy without adhesion. In the other groups, the kidney was hypertrophic and grey in colour, similar to large white kidney seen in human. There were granules on the surface of the kidney, and kidney peplos were tacky in some areas.

□ Light Microscopic Examination:

In the sham operation group, nephrons had very clear structure. Dilation or atrophy of glomerular capsule was not observed. No degeneration or necrosis of tubular epithelial cells was seen. There were no exfoliative epithelial cells or tubules. No vasodilation or inflammatory cell infiltration were seen in interstitial tissue. In the model group, much tubular necrosis and dilation were observed, with a large amount of buffy-colored refractive substances or necrotic and exfoliative epithelial cells in the tubular cavity. Interstitial fibrocytes proliferated, and the number of glomeruli declined. Some glomeruli were fibrotic and adhesive to a glomerular capsule, with glomerular cavity disappearing. RA treated animals showed improvement of various degrees in lesions, with significant difference from the model group.

As shown in table 7, i.v. dose of RA at 2.5mg/kg, 5 mg/kg, 25 mg/kg, 75 mg/kg, and 150 mg/kg and p.o. dose of 5 mg/kg, 25 mg/kg, 150 mg/kg, and 300 mg/kg can all significantly reduce FN and HYP levels (compared with the model group, p<0.05or 0.01).

TABLE 7
Effect of RA on renal interstitial fibrosis in rats
with unilateral urethral obstruction ( x̅ ± SD, n = 10)

| Group | Dosage (mg/kg) | HYP (μg/g) | FN (mg/L) |
|---|---|---|---|
| Sham operation group | — | 320 ± 53** | 5.2 ± 1.3** |
| Model group | — | 788 ± 157 | 29.6 ± 5.8 |
| Benazapril group | 10 | 500 ± 132 | 17.0 ± 3.6** |
| RA i.v. group | 2.5 | 612 ± 102* | 22.7 ± 6.0* |
| | 5 | 605 ± 118* | 19.9 ± 6.3* |
| | 25 | 573 ± 98** | 17.7 ± 6.2** |
| | 75 | 533 ± 94** | 16.5 ± 4.3** |
| | 150 | 526 ± 121** | 16.4 ± 4.4** |
| RA p.o. group | 2.5 | 705 ± 162 | 22.4 ± 6.5 |
| | 5 | 612 ± 118* | 19.8 ± 6.2* |
| | 25 | 573 ± 98** | 17.5 ± 6.3** |
| | 150 | 563 ± 94** | 17.2 ± 4.3** |
| | 300 | 556 ± 121** | 17.2 ± 4.4** |

*P < 0.05 and
**P < 0.01 compared with the model group.

### Test Example 5

### Effect of RA on CRF Induced by Nephrectomy in Rats

5.1 Drugs and Reagents

RA formulations were prepared according to the Examples 1 and 2.

Methylprednisolone (40 mg/vial) was provided by Pharmacia Upjohn, Belgium; creatinine and urea nitrogen detection kits were purchased from Beijing Biosino Bio-technology and Science Inc.

Experimental animals: Male SPF Sprague Dawley rats weighing 220 g-250 g were provided by Experimental Animal Centre of Shangdong Luye Phaimaceuticals Co. Ltd. Animal certificate number is SYXK (Lu) 20030020.

5.2 Methods and Results:

130 rats were randomly divided into 13 groups, with 10 animals in each group, namely, a normal control group, a model group, methylprednisolone p.o. Group at 12 mg/kg, RA i.v. groups at 2.5 mg/kg, 5 mg/kg, 25 mg/kg, 75 mg/kg, and 150 mg/kg of RA, and RA p.o. groups at 5 mg/kg, 10 mg/kg, 50 mg/kg, 150 mg/kg, and 300 mg/kg of RA. Apart from the normal control group, all other rats were induced to CRF by cutting off 5/6 of the kidneys. 2/3 of the left kidney was removed in the first operation, and one week later the right kidney in the second operation. Dosing started one week after the second operation via p.o. or i.p. routes. Blood was collected from eyepit at week 4 and week 8 to determine creatinine and urea nitrogen levels in sera. 5 animals in each group were sacrificed at week 4 and all 10 were sacrificed at the end of the study. Pathological changes in renal function were observed through HE and PAS staining of histopathological sections.

TABLE 8
Effect of RA on serum creatinine and urea nitrogen levels in CRF rats
induced by ⅚ nephrectomy after 4 weeks of treatment with RA
( x̅ ± SD, n = 10)

| | Dosage | Serum creatinine | Serum urea nitrogen |
|---|---|---|---|
| Group | (mg/kg) | (mg/dL) | (mg/dL) |
| Normal control group | — | 0.50 ± 0.05** | 9.05 ± 2.52** |
| Model group | — | 1.92 ± 0.19 | 28.54 ± 2.53 |
| Methylprednisolone | 12 | 1.33 ± 0.13* | 15.21 ± 3.25** |
| group | | | |
| RA i.v. group | 2.5 | 1.32 ± 0.18* | 21.12 ± 2.40* |
| | 5 | 1.29 ± 0.31** | 16.90 ± 4.09** |
| | 25 | 1.12 ± 0.24** | 14.63 ± 3.25** |
| | 75 | 1.02 ± 0.19** | 13.39 ± 2.45** |
| | 150 | 1.01 ± 0.33** | 12.85 ± 3.88** |
| RA p.o. group | 5 | 1.55 ± 0.16* | 20.21 ± 2.22** |
| | 10 | 1.42 ± 0.24** | 18.60 ± 3.11** |
| | 50 | 1.53 ± 0.15* | 19.95 ± 2.20* |
| | 150 | 1.30 ± 0.22** | 17.01 ± 3.95** |
| | 300 | 1.29 ± 0.28** | 16.76 ± 4.30** |

*P < 0.05 and
**P < 0.01 compared with the model group.

TABLE 9
Effect of RA on serum creatinine and urea nitrogen levels in CRF rats
induced by ⅚ nephrectomy after 8 weeks of treatment with RA
( x̅ ± SD, n = 10)

| | Dosage | Serum creatinine | Serum urea nitrogen |
|---|---|---|---|
| Group | (mg/kg) | (mg/dL) | (mg/dL) |
| Normal control group | — | 0.7 ± 0.08 | 9.05 ± 2.32 |
| Model group | — | 2.93 ± 0.26 | 38.98 ± 3.25 |
| Methylprednisolone | 12 | 1.25 ± 0.17** | 18.21 ± 4.25** |
| group | | | |
| RA i.v. group | 2.5 | 1.89 ± 0.22* | 22.15 ± 3.09* |
| | 5 | 1.75 ± 0.37** | 21.73 ± 5.26** |
| | 25 | 1.51 ± 0.32** | 19.25 ± 3.19** |
| | 75 | 1.39 ± 0.26** | 15.07 ± 3.45** |
| | 150 | 1.37 ± 0.44** | 14.85 ± 3.88** |
| RA p.o. group | 5 | 2.09 ± 0.22* | 25.98 ± 2.72** |
| | 10 | 2.06 ± 0.21* | 25.65 ± 2.57* |
| | 50 | 1.92 ± 0.32** | 23.91 ± 4.00** |
| | 150 | 1.76 ± 0.30** | 21.96 ± 3.95** |
| | 300 | 1.74 ± 0.36** | 20.76 ± 5.50** |

*P < 0.05 and
**P < 0.01 compared with the model group.

As shown in table 8 and 9, i.v. dose of RA at 2.5mg/kg, 5 mg/kg, 25 mg/kg, 75 mg/kg, and 150 mg/kg and p.o. dose of RA at 5 mg/kg, 10 mg/kg, 50 mg/kg, 150 mg/kg, and 300 mg/kg can all significantly reduce serum creatinine and urea nitrogen levels in CRF rats compared with the model group.

Pathological Changes:

At week 4, glomerular compensatory hypertrophy of various degrees were observed under light microscope in the model group, together with masengial cell proliferation, more masengial matrix, and phyllode glomerular capillary loops. Some glomerular cavity disappeared and capillary wall thickened. Tubules were swelling or dilating, with protein deposition in the cavity. Some tubules had atrophy, with inflammatory cell infiltration in interstitial tissue. All these lesions were alleviated in RA treated animals in a dose-dependent manner. At week 8, the model group showed proliferation of masengial cells and more matrix accumulation under light microscope. Capillary walls were collapsed, with segmental cirrhosis which was mostly found close to the vascular wall. Homogenous dark-red round deposits (PAS staining) were seen in cirrhotic glomeruli. Focal glomerulus-capsule adhesion was observed in some glomeruli with focal crescent formation. Inflammatory cell infiltration was observed in interstitial tissue with fibrous tissue hyperplasia. All these lesions were alleviated by RA treatment in a dose-dependent manner.

### Test Example 6

### Effect of RA on Rats with Diabetic Nephropathy

6.1 Materials

RA formulations were prepared according to the Examples 1 and 2.

Benazapril was provided by Beijing Novartis Pharmaceuticals; streptozocin was from Sigma; blood glucose test kit was obtained from Beijing Biosino Reagents Inc; creatinine and urea nitrogen detection kits were purchased from Beijing Biosino Bio-technology and Science Inc.

Experimental animals: Male SPF Sprague Dawley rats weighing 220 g-250 g were provided by Experimental Animal Centre of Shangdong Luye Pharmaceuticals Co. Ltd. Animal certificate number is SYXK (Lu) 20030020.

6.2 Methods and Results:

After 1 week-long acclimation period, animals were anaesthetised with 10% chloral hydrate (0.35 mL/100 g, i.p.) and nephrectomy was exercised to the left kidney. One week later, a single dose of 1% of streptozocin solution was i.v. injected to all animals at 60 mg/kg. After 72h, blood was collected from eyepit to test blood glucose level with the above-mentioned kit. The model was considered successful when blood glucose level >16.7 mmol/L. When the model is established, 130 rats were randomly divided into 13 groups, with 10 animals in each group, namely, a normal control group, a model group, benzanapril p.o. Group at 10 mg/kg, RA i.v. groups at 2.5 mg/kg, 5 mg/kg, 25 mg/kg, 75 mg/kg, and 150 mg/kg of RA, and RA p.o. groups at 5 mg/kg, 10 mg/kg, 50 mg/kg, 150 mg/kg, and 300 mg/kg of RA. The drugs were administered once every day. Blood was collected from eyepit at week 8 and week 16 to determine serum levels of creatinine and urea nitrogen. Urinary protein excretion within 24 hours was also studied. The animals were sacrificed 24 hours after the last dose and kidney removed for histopathological examination.

As shown in table 10 and 11, i.v. dose of RA at 2.5mg/kg, 5 mg/kg, 25 mg/kg, 75 mg/kg, and 150 mg/kg and p.o. dose of RA at 5 mg/kg, 10 mg/kg, 50 mg/kg, 150 mg/kg, and 300 mg/kg can all significantly reduce serum creatinine and urea nitrogen levels as well as 24-hour urinary protein excretion at week 8 and week 16.

TABLE 10
Effect of RA on rats with diabetic nephropathy after 8 weeks of
treatment with RA ( x̅ ± SD, n = 10)

| | | | Serum urea | 24 h urinary protein | Blood |
|---|---|---|---|---|---|
| | Dosage | creatinine | nitrogen | excretion | glucose |
| Group | (mg/kg) | (mg/dL) | (mg/dL) | (mg/24 h) | (mmol/L) |
| Normal | — | 0.47 ± 0.05** | 9.95 ± 2.55** | 5.21 ± 1.42** | 5.25 ± 0.33** |
| control | | | | | |
| group | | | | | |
| Model group | — | 1.53 ± 0.23 | 27.57 ± 4.55 | 26.32 ± 3.86 | 23.45 ± 5.34 |
| Benazapril | 10 | 0.57 ± 0.07** | 9.21 ± 1.62** | 19.29 ± 2.11* | 21.11 ± 4.55 |
| group | | | | | |
| RA i.v. | 2.5 | 0.91 ± 0.12* | 18.01 ± 1.62* | 20.29 ± 2.21* | 21.23 ± 5.66 |
| group | 5 | 0.90 ± 0.09* | 17.91 ± 1.80* | 19.19 ± 2.15* | 21.02 ± 6.14 |
| | 25 | 0.75 ± 0.18** | 15.42 ± 3.36** | 17.59 ± 2.12** | 21.15 ± 5.74 |
| | 75 | 0.72 ± 0.15** | 14.76 ± 2.19** | 16.78 ± 2.52** | 21.03 ± 4.74 |
| | 150 | 0.69 ± 0.11** | 13.87 ± 1.75** | 16.39 ± 2.25** | 21.23 ± 5.54 |
| RA p.o. | 5 | 0.96 ± 0.09 | 19.37 ± 3.85 | 20.59 ± 2.34 | 21.23 ± 6.36 |
| group | 10 | 0.88 ± 0.15* | 17.57 ± 2.70* | 19.11 ± 2.19* | 20.87 ± 6.55 |
| | 50 | 0.77 ± 0.13** | 15.96 ± 1.95** | 17.89 ± 2.64** | 21.07 ± 5.46 |
| | 150 | 0.75 ± 0.12** | 14.76 ± 2.65** | 17.18 ± 2.56** | 21.32 ± 6.44 |
| | 300 | 1.40 ± 0.28** | 14.42 ± 2.90** | 16.89 ± 2.75** | 20.97 ± 5.54 |

*P < 0.05 and
**P < 0.01 compared with the model group.

TABLE 11
Effect of RA on rats with diabetic nephropathy after 16 weeks of
treatment with RA ( x̅ ± SD, n = 10)

| | | | Serum urea | 24 h urinary protein | Blood |
|---|---|---|---|---|---|
| | Dosage | creatinine | nitrogen | excretion | glucose |
| Group | (mg/kg) | (mg/dL) | (mg/dL) | (mg/24 h) | (mmol/L) |
| Normal | — | 0.48 ± 0.05** | 10.05 ± 2.52** | 5.45 ± 1.49** | 5.43 ± 0.33** |
| control group | | | | | |
| Model group | — | 2.43 ± 0.23 | 48.57 ± 4.55 | 77.36 ± 12.35 | 23.45 ± 5.54 |
| Benazapril | 10 | 1.15 ± 0.13** | 18.21 ± 3.25** | 61.76 ± 6.75* | 22.11 ± 4.55 |
| group | | | | | |
| RA i.v. group | 2.5 | 1.60 ± 0.18* | 33.91 ± 3.60* | 60.96 ± 6.85* | 22.02 ± 5.34 |
| | 5 | 1.52 ± 0.37** | 30.42 ± 7.36** | 57.28 ± 8.46** | 22.15 ± 4.24 |
| | 25 | 1.32 ± 0.28** | 26.76 ± 4.19** | 55.34 ± 7.67** | 22.03 ± 4.59 |
| | 75 | 1.26 ± 0.28** | 23.07 ± 3.95** | 54.78 ± 7.96** | 22.35 ± 7.28 |
| | 150 | 1.20 ± 0.22** | 22.57 ± 3.45** | 54.28 ± 7.46** | 22.23 ± 6.24 |
| RA p.o. group | 5 | 1.97 ± 0.19 | 39.37 ± 3.85 | 65.55 ± 8.71 | 22.23 ± 5.66 |
| | 10 | 1.92 ± 0.19* | 38.37 ± 3.85** | 61.33 ± 6.65* | 21.87 ± 5.55 |
| | 50 | 1.77 ± 0.28* | 34.57 ± 5.60* | 57.28 ± 8.46** | 22.07 ± 4.46 |
| | 150 | 1.53 ± 0.28** | 28.96 ± 3.95** | 56.28 ± 8.76** | 21.32 ± 6.34 |
| | 300 | 1.50 ± 0.26** | 27.76 ± 5.30** | 55.78 ± 9.46** | 21.97 ± 5.34 |

*P < 0.05 and
**P < 0.01 compared with the model group.

Pathological Examination:

Normal control animals had normal glomerular basement membrane (GBM) with well-aligned podocytic processes. Model animals had thicker GBM with irregular podocytic processes some of which were fused together. Mild segmental proliferation of magengial cells and matrix were also observed. RA treated groups showed fewer lesions than the model group in a dose-dependent manner.

### INDUSTRIAL APPLICATION

The present invention demonstrated through experiments that RA can inhibit CTGF expression and plays an important role in inhibiting the development and progress of hepatofibrosis and nephrofibrosis. Therefore, RA can be' used in the manufacture of medicaments useful for the prevention or treatment of chronic hepatitis, CRF, and diabetic nephropathy. The present compositions can be administered directly to exert marked efficacy and therefore are promising to become medicaments useful for the prevention or treatment of chronic hepatitis, CRF, and diabetic nephropathy.

1. Use of Rosmarinic acid as a connective tissue growth factor inhibitor.

2. Use of Rosmarinic acid in the manufacture of medicaments useful for the prevention or treatment of diseases related to abnormal expression of connective tissue growth factor.

3. The use according to claim 2, wherein said diseases related to abnormal expression of connective tissue growth factor include but are not limited to hepatofibrosis and nephrofibrosis.

4. The use according to claim 2, which is the use of Rosmarinic acid in the manufacture of medicaments useful for the manufacture or treatment of chronic hepatitis.

5. The use according to claim 2, which is the use of Rosmarinic acid in the manufacture of medicaments useful for the prevention or treatment of diabetic nephropathy.

6. The use according to claim 2, which is the use of Rosmarinic acid in the manufacture of medicaments useful for the prevention or treatment of chronic renal failure.

7. A pharmaceutical composition, comprising of an effective amount of Rosmarinic acid in the prevention or treatment of diseases related to abnormal expression of connective tissue growth factor, and pharmaceutically acceptable carriers or excipients.

8. The pharmaceutical composition according to claim 7, wherein said effective amount of Rosmarinic acid are in the range of 25-1,500 mg, preferably 25-750 mg when injected; 50 mg-3,000 mg, preferably 50-1,500 mg when taken orally.

9. The pharmaceutical composition according to claim 7, which is in the form of tablets, capsules, pills, solutions for injection, freeze-dried powers, or emulsions for injection, preferably tablets, pills, or freeze-dried powders.

10. The pharmaceutical composition according to claim 9, which is prepared by the following steps: 2,000 volume unit of water for injection use is added to 50.0 weigh unit of Rosmarinic acid to dissolve it; add NaOH until pH=5.5˜7.5; add 8 weigh unit of mannitol, and stir until it completely dissolves;
pyrogen-free clear solution is obtained after ultrafiltration and 2 ml of the solution is filled into each 10 ml tube vials; lyophilisation process is carried out to obtain lyophilised preparations with 50.0 mg of Rosmarinic acid in each vial.

11. The pharmaceutical composition according to claim 9, which is prepared by the following steps: 100.0 weigh unit of Rosmarinic acid, 35.0 weigh unit of sucrose, 40.0 weigh unit of lactose, and 23.0 weigh unit of Sodium Carboxymethyl Starch are mixed well and passed through 100-mesh sieve; 3% of PVP_{K30} solution is added as needed to make a damp mass, which is then granulated through 20-mesh sieve; after dried at 60° C. for 3 hours, the granules are passed through 18-mesh sieve; 2.weight unit of magnesium stearate is added and mixed completely before compressed with shallow concavity to obtain tablets weighing about 200 mg and containing 100 mg of Rosmarinic acid each.

12. The pharmaceutical composition according to claim 8, which is in the form of tablets, capsules, pills, solutions for injection, freeze-dried powers, or emulsions for injection, preferably tablets, pills, or freeze-dried powders.

13. The pharmaceutical composition according to claim 12, which is prepared by the following steps: 2,000 volume unit of water for injection use is added to 50.0 weigh unit of Rosmarinic acid to dissolve it; add NaOH until pH=5.5˜7.5; add 8 weigh unit of mannitol, and stir until it completely dissolves; pyrogen-free clear solution is obtained after ultrafiltration and 2 ml of the solution is filled into each 10 ml tube vials; lyophilisation process is carried out to obtain lyophilised preparations with 50.0 mg of Rosmarinic acid in each vial.

14. The pharmaceutical composition according to claim 12, which is prepared by the following steps: 100.0 weigh unit of Rosmarinic acid, 35.0 weigh unit of sucrose, 40.0 weigh unit of lactose, and 23.0 weigh unit of Sodium Carboxymethyl Starch are mixed well and passed through 100-mesh sieve; 3% of PVP_{K30} solution is added as needed to make a damp mass, which is then granulated through 20-mesh sieve; after dried at 60° C. for 3 hours, the granules are passed through 18-mesh sieve; 2.weight unit of magnesium stearate is added and mixed completely before compressed with shallow concavity to obtain tablets weighing about 200 mg and containing 100 mg of Rosmarinic acid each.
